(19) United States
(12) Patent Application Publication
(10) Pub. No.: US 2012/0220663 A1
(43) Pub. Date: Aug. 30, 2012

(54) SOLID FORMS OF ALISKIREN HEMIFUMARATE AND PROCESSES FOR PREPARATION THEREOF

(75) Inventors: Marina Etinger, Nesher (IL); Rinat Moshkovits Kaptsan, Raanana (IL); Ariel Mittelman, Elad (IL)

(73) Assignees: TEVA PHARMACEUTICALS USA, INC., North Wales, PA (US); TEVA PHARMACEUTICAL INDUSTRIES LTD., Petah Tiqva (IL)

(21) Appl. No.: 13/393,949

(22) PCT Filed: Sep. 2, 2010
(86) PCT No.: PCT/US2010/047701

§ 371 Date: May 11, 2012

Related U.S. Application Data
(60) Provisional application No. 61/239,554, filed on Sep. 3, 2009.
(60) Provisional application No. 61/244,339, filed on Sep. 21, 2009.
(60) Provisional application No. 61/263,754, filed on Nov. 23, 2009.
(60) Provisional application No. 61/264,498, filed on Nov. 25, 2009.
(60) Provisional application No. 61/370,921, filed on Aug. 5, 2010.

(51) Int. Cl. A61K 31/165; A61P 9/12; C07C 237/22; C07C 231/22

(52) U.S. Cl. 514/616; 564/157

(57) ABSTRACT

The present invention provides polymorphic forms of aliskiren hemifumarate, and processes for preparation thereof and for the preparation of the amorphous form of aliskiren hemifumarate. The present invention also provides pharmaceutical compositions comprising the aliskiren hemifumarate crystalline forms T1, T3 or T4, T5, T6, T7, T8 and at least one pharmaceutically acceptable excipient, and the use of these pharmaceutical compositions in the treatment of hypertension.

### CROSS REFERENCE TO RELATED APPLICATIONS

This application claims the benefit of U.S. Provisional Application Nos. 61/239,554, filed Sep. 3, 2009; 61/244,339, filed Sep. 21, 2009; 61/263,754, filed Nov. 23, 2009; 61/264,498, filed Nov. 25, 2009; and 61/370,921, filed Aug. 5, 2010, herein incorporated by reference in its entirety.

### FIELD OF THE INVENTION

The present invention relates to solid forms of aliskiren hemifumarate, and processes for preparing said forms.

### BACKGROUND OF THE INVENTION

Aliskiren hemifumarate [CAS Registry Number: 173334-58-2], having the chemical name: (2S, 4S, 5S, 7S)—N-(2-carbamoyl-2-methylpropyl)-5-amino-4-hydroxy-2,7-diisopropyl-8-[4-methoxy-3-(3-methoxypropoxy)phenyl]octanamide hemifumarate [C₃₀H₅₃N₃O₆.0.5C₄H₄O₄] and the following structure:

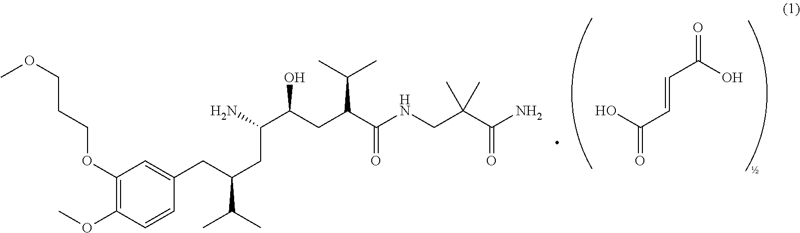

is indicated for treatment of hypertension, acting as a renin inhibitor, and marketed by Novartis as TEKTURNA® in a once-daily formulation. Aliskiren and its related compounds are referred to in U.S. Pat. No. 5,559,111, while synthesis, pharmacological actions, pharmacokinetics and clinical studies of aliskiren and its related compounds are referred to in Lindsay, K. B. et al, J. Org. Chem., Vol. 71, pp 4766-4777 (2006) and in Drugs of the Future, Vol. 26, No. 12, pp 1139-1148 (2001).

U.S. Pat. No. 5,559,111 refers to the preparation of a crystalline form of aliskiren hemifumarate having a melting point of about 95-104° C. by crystallizing from an ethanol/acetonitrile mixture in a 1 to 19 volume ratio and then drying at 60° C.

U.S. Pat. No. 6,730,798 refers to the preparation of aliskiren hemifumarate from aliskiren base and fumaric acid in ethanol/acetonitrile.

WO 2008/061622 (“WO '622”) describes an amorphous form and several crystalline fauns including Modification A, Modification B, Solvate S_{A} (also called form E), Solvate S_{B}, Solvate S_{C}, Solvate S_{D}, Type I, Type II, Type III, Type IV, Type V and Type VI of aliskiren hemifumarate.

WO 2009/0064479 describes a stable amorphous form and crystalline forms including Forms I, II, III, V, VII, VIII, IX, and X of aliskiren hemifumarate.

WO 2005/089729 (“WO '729”) refers to solid oral dosage fauns comprising aliskiren obtained by a process comprising wet granulation of the API, drying the obtained granulate, mixing with an outer phase excipient and further compressing to obtain a tablet. WO '729 discusses the difficulties in formulation of aliskiren due to the needle-shaped habit of its crystals. Moreover, it claims that the compression behavior of the drug substance is poor and therefore direct compression is a difficult option for routine production.

This patent application also refers to the difficulties in formulation of aliskiren. The hurdles include the high hygroscopicity of aliskiren, its relatively low stability and the variability in drug substance quality. The latter has effects on the processability of a tablet, leading to a more complicated manufacturing process, in particular when isolating the final product.

The present invention relates to the solid state physical properties of aliskiren hemifumarate. These properties can be influenced by controlling the conditions under which aliskiren hemifumarate is obtained in solid foam. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic foams from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms of aliskiren hemifumarate provides a new opportunity to improve the performance of the synthesis of the active pharmaceutical ingredient (API), aliskiren hemifumarate, by producing polymorphs of aliskiren hemifumarate having improved characteristics, such as flowability and solubility. Thus, there is a need in the art for solid forms of aliskiren hemifumarate.

### SUMMARY OF THE INVENTION

The present invention provides new crystalline forms of aliskiren hemifumarate

The invention further provides a pharmaceutical formulation comprising the below described crystalline forms of aliskiren hemifumarate. This pharmaceutical composition may additionally comprise at least one pharmaceutically acceptable excipient.

The invention further provides the use of the solid state forms described below for the manufacture of a medicament for the treatment of hypertension.

### BRIEF DESCRIPTION OF THE FIGURES

FIG. 1 provides a powder XRD pattern of crystalline aliskiren hemifumarate form T1 prepared according to example 2.

FIG. 2 provides a powder XRD pattern of crystalline aliskiren hemifumarate form T3 prepared according to example 3.

FIG. 3 provides a powder XRD pattern of crystalline aliskiren hemifumarate form T4 prepared according to example 3.

FIG. 4 provides a powder XRD pattern of crystalline aliskiren hemifumarate Bann T4 prepared according to example 4.

FIG. 5 provides a powder XRD pattern of crystalline aliskiren hemifumarate form T5 prepared according to example 8.

FIG. 6 provides a powder XRD pattern of crystalline aliskiren hemifumarate faun T5 prepared according to example 9.

FIG. 7 provides a powder XRD pattern of crystalline aliskiren hemifumarate form T6 prepared according to example 10.

FIG. 8 provides a powder XRD pattern of crystalline aliskiren hemifumarate form T7 prepared according to example 11.

FIG. 9 provides a powder XRD pattern of crystalline aliskiren hemifumarate form T8 prepared according to example 12.

### DETAILED DESCRIPTION OF THE INVENTION

The invention addresses a need in the art by providing crystalline forms of aliskiren hemifumarate and processes for their preparation.

A crystal form may be referred to herein as being characterized by graphical data “as shown in” a Figure. Such data include, for example, powder X-ray diffractograms and solid state NMR spectra. The skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

As used herein, Aliskiren hemifumarate Form I refers to a crystalline aliskiren hemifumarate characterized by a powder XRD pattern with peaks at 3.8, 6.6, 7.6, 8.0, 13.8, 14.5, 15.6 and 17.4±0.2 degrees 2-theta. This form can be prepared as indicated in PCT application No. PCT/US08/012816.

As used herein, Aliskiren hemifumarate Form VIII refers to a crystalline aliskiren hemifumarate characterized by a powder XRD pattern having peaks at 6.0, 7.4, 9.3 and 11.1±0.2 degrees two theta. This form can be prepared as indicated in PCT application No. PCT/US08/012816.

In one embodiment the present invention provides a crystalline form of aliskiren hemifumarate, designated Form T8.

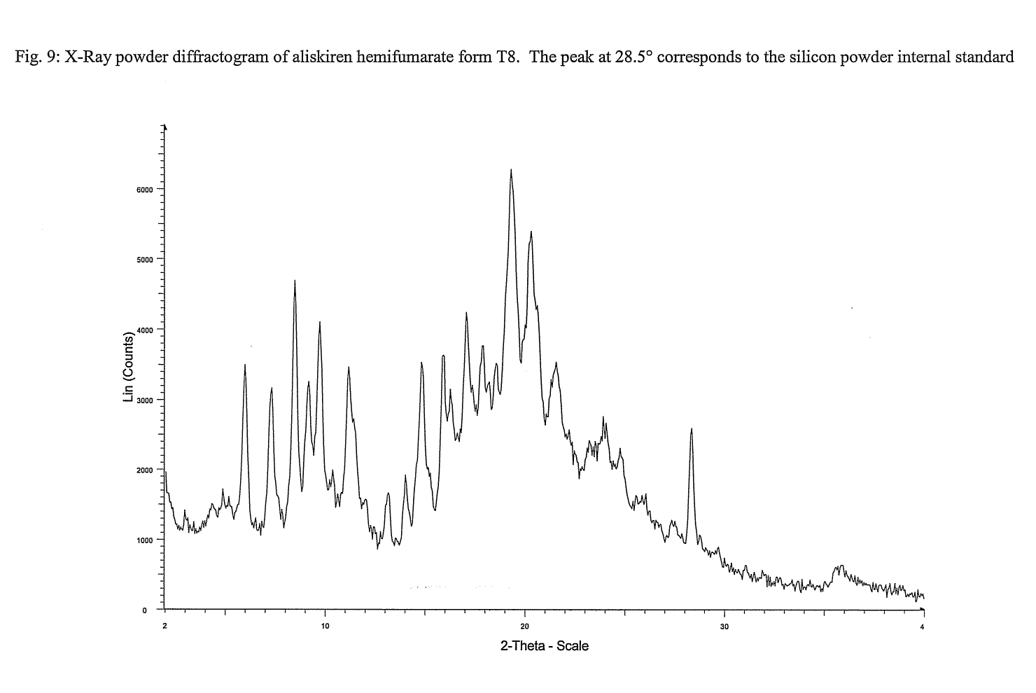

Form T8 can be characterized by data selected form: a PXRD pattern having peaks at 13.3°, 14.1°, 18.7°, 19.4° and 20.4°±0.2 degrees 2-theta and free from peak at 20.1°±0.2 degrees 2-theta; a PXRD pattern having peaks at 13.3°, 14.1°, 18.7°, 19.4° and 20.4°±0.2 degrees 2-theta and additional peaks at 9.8°, 10.5° and 11.3°±0.2 degrees 2-theta; a powder XRD pattern as shown in FIG. 9.

In another embodiment the present invention provides aliskiren hemifumarate form T8 containing 20% (w/w) or less, 10% (w/w) or less, 5% (w/w) or less, 2% (w/w) or less, particularly 1% (w/w) or less, more particularly 0.5% (w/w) or less, and most particularly 0.2% (w/w) or less of any other polymorph, for example Form VIII. For examples, T8 of the invention contains from 0.1% to 5% (w/w), from 0.2% to 5% (w/w), or from 0.2% to 2% (w/w) of the any other polymorph, for example, Form VIII. In other embodiments, the T8 of the invention contains from 0.1% to 20% (w/w), from 1% to 20% (w/w), from 5% to 20% (w/w), or from 5% to 10% (w/w) of any other polymorph, for example Form VIII.

Aliskiren hemifumarate Form T8 can be characterized by any combination of the above data.

In another embodiment, the present invention provides a process for preparing aliskiren hemifumarate form T8 comprising comminuting aliskiren hemifumarate form VIII and drying. Comminuting is preferably conducted by using a granulator.

Drying is preferably performed by heating to a temperature of about 40° C. to about 65° C. for a time from about 45 hours to about 150 hours. Preferably, heating is performed in two stages. In the first stage, heating is up to a temperature of about 40° C. to about 50° C., more preferably to about 45° C., for about 45 to 55 hours, more preferably for about 45 hours. The second stage is up to a temperature of about 55° C. to about 65° C., more preferably to about 60° C., for about an additional 55 to about 65 hours, more preferably for about 60 hours.

In another embodiment; the present invention encompasses a number of methods for preparing the amorphous aliskiren hemifumarate.

According to one method, the amorphous aliskiren hemifumarate may be prepared by a process comprising suspending aliskiren hemifumarate form T3 in DEKALIN® or amyl alcohol to obtain amorphous aliskiren hemifumarate, and recovering amorphous aliskiren hemifumarate from the suspension. Preferably, when DEKALIN® is used, the suspension is maintained at about room temperature for a period of time sufficient to obtain the amorphous aliskiren hemifumarate. Preferably, the suspension is maintained for a period of about 10 hours to about 20 hours. More preferably, for a period of about 10 to about 15 hours and most preferably for about 12 hours.

Preferably, when amyl alcohol is used, the suspension is maintained at a temperature of about 40° C. to about 65° C., more, preferably at about 60° C. and then cooled to room temperature. Preferably, the suspension is maintained at a temperature of about 40° C. to about 65° C. for a period of about 10 hours to about 20 hours; more preferably, for a period of about 10 to about 15 hours.

In one specific embodiment, aliskiren hemifumarate form T3 is suspended in amyl alcohol for a period of about 12 hours at 60° C. and then cooled to room temperature. Following the cooling step, diethyl ether is added and the suspension is maintained at room temperature for an additional 2 hours to obtain a precipitate.

According to another method, amorphous aliskiren hemifumarate may be prepared by providing a solution of aliskiren hemifumarate in an organic solvent selected from benzyl alcohol, methyl formate, dimethylacetamide, N-methyl-2-pyrrolidone (NMP) and mixtures thereof, and further combining with methyl tent butyl ether (MTBE) to obtain amorphous aliskiren hemifumarate.

Optionally, prior to addition of MTBE, hexane is added to the solution of aliskiren hemifumarate. Preferably, when hexane and MTBE are added, they are added in a volume ratio of about 1:1 (v/v).

Preferably, the ratio between aliskiren hemifumarate and the organic solvent used in the process described above is about 1:3 (w/v) to about 1:4 (w/v) of grams of aliskiren hemifumarate to milliliters of 2-methyltetrahydrofuran, more preferably, the ratio is about 1:4 (w/v).

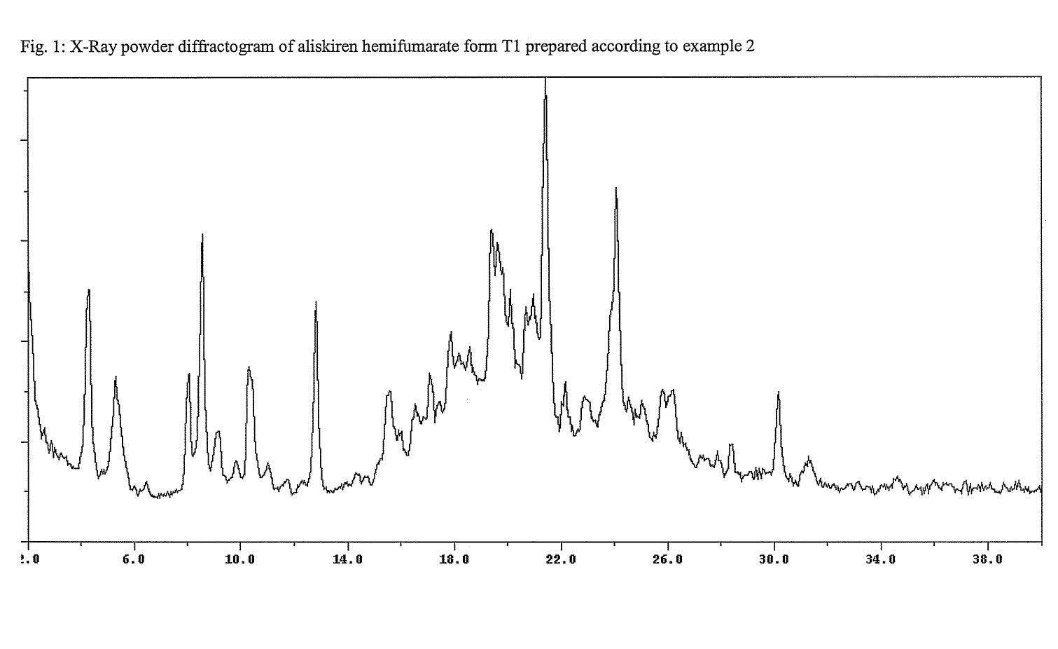

The present invention also provides a crystalline form of aliskiren hemifumarate, designated faun T1, characterized by an XRPD pattern with peaks at 4.3, 8.5, 12.8, 21.4 and 24.1±0.2 degrees 2-theta, and optionally additional peaks at 5.3, 8.0, 10.3, 15.5 and 19.4±0.2 degrees 2-theta, as occur in the X-ray diffractogram depicted in FIG. 1.

Aliskiren hemifumarate form T1 can be prepared by a process comprising slurrying amorphous aliskiren hemifumarate in 2-methyltetrahydro-furan, wherein slurrying is performed for a period of about 48 to about 75 hours. Preferably, slurrying is for a period of about 65 to about 75 hours, more preferably for about 72 hours. Preferably, the ratio between amorphous aliskiren hemifumarate and 2-methyltetrahydrofuran is about 1:3 (w/v) to about 1:4 (w/v) of gram aliskiren hemifumarate to ml 2-methyltetrahydrofuran; more preferably the ratio is about 1:4 (w/v).

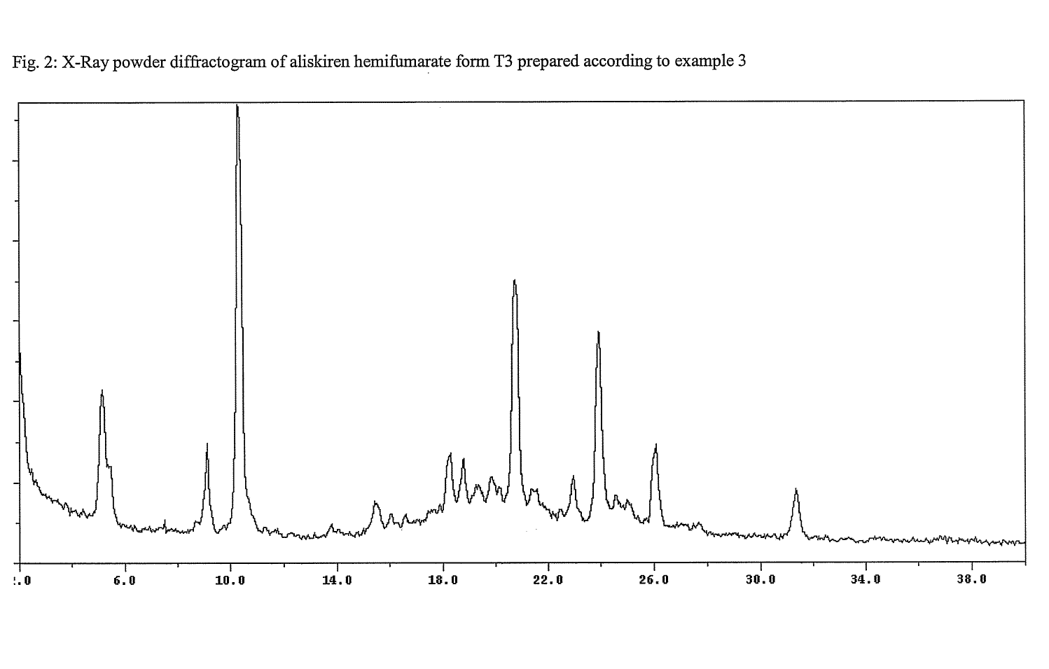

The present invention also provides a crystalline form of aliskiren hemifumarate, designated form T3, characterized by an XRPD pattern with peaks at 5.1, 9.1, 10.3, 15.5 and 18.3±0.3 degrees 2-theta and optionally additional peaks at 5.4, 18.8, 20.8, 22.9 and 23.9±0.3 degrees 2-theta as occur in the X-ray diffractogram depicted in FIG. 2.

Aliskiren hemifumarate form T3 can be prepared by a process comprising slurrying amorphous aliskiren hemifumarate in 2-methyltetrahydro-furan, wherein slurrying is performed for a period of about 30 minutes to about 2 hours. Preferably, slurrying is for a period of about 30 minutes to about 90 minutes, more preferably for about 1 hour.

Preferably, the ratio between amorphous aliskiren hemifumarate and 2-methyltetrahydrofuran is about 1:5 (w/v) to about 1:7 (w/v) of grams of aliskiren hemifumarate to milliliters of 2-methyltetrahydrofuran. More preferably the ratio is about 1:6.6 (w/v).

Slurrying is preferably performed at a temperature of about 15° C. to about 25° C., more preferably at about 20° C. to about 25° C., and most preferably at about 25° C.

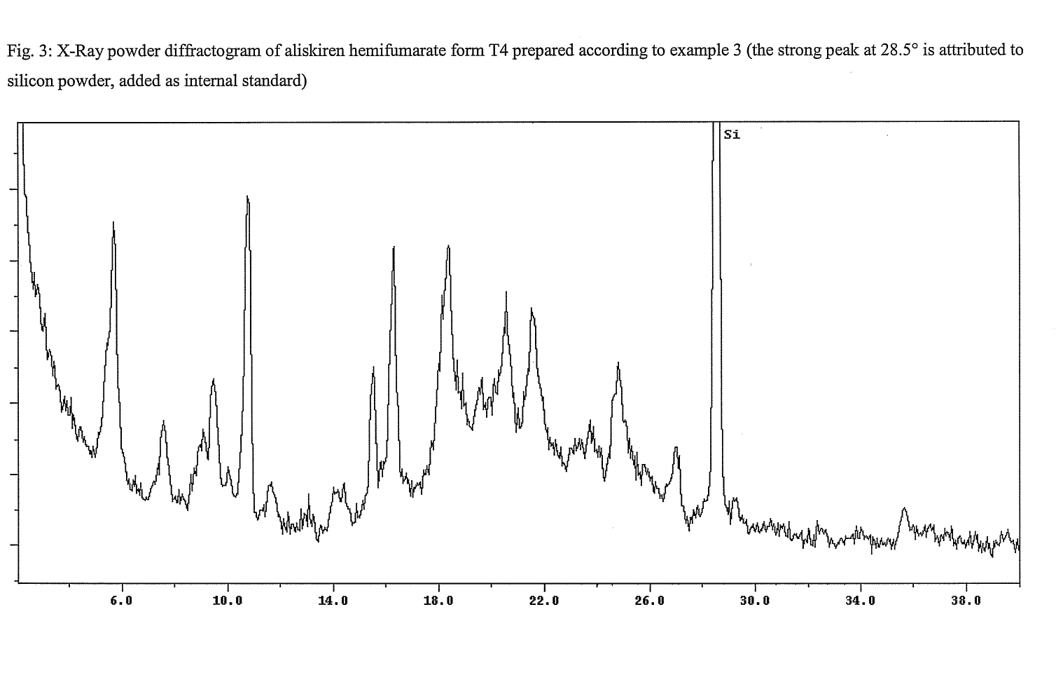

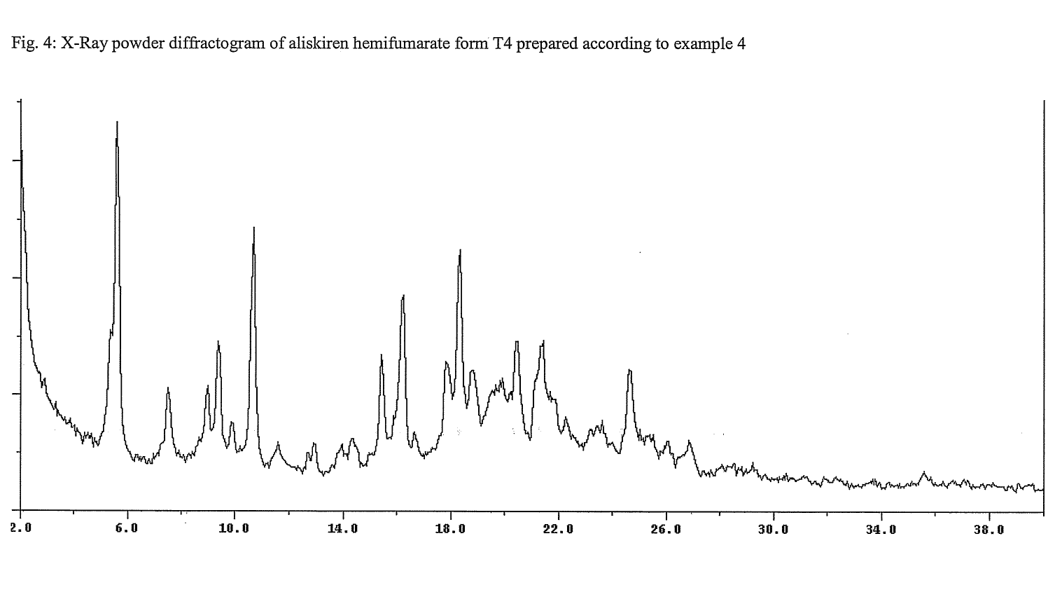

The present invention also provides a crystalline faun of aliskiren hemifumarate, designated than T4, characterized by an XRPD pattern with peaks at 5.6, 9.4, 10.6, 15.4 and 16.2±0.2 degrees 2-theta, and optionally additional peaks at 7.5, 8.9, 18.3, 20.4 and 24.6±0.2 degrees 2-theta as occur in the X-ray diffractogram depicted in FIGS. 3 and 4.

Aliskiren hemifumarate form T4 can be prepared for example by drying aliskiren hemifumarate form T1 or T3. Drying is performed at about 45° C. to about 60° C., more preferably at about 50° C. to about 60° C., and most preferably at about 55° C. to about 60° C. Preferably, the drying is performed under vacuum (a pressure of less than 100 mm Hg). Drying can be performed for at least about 10 hours, for example, about 48 hours. More preferably, drying is performed for about 12 hours.

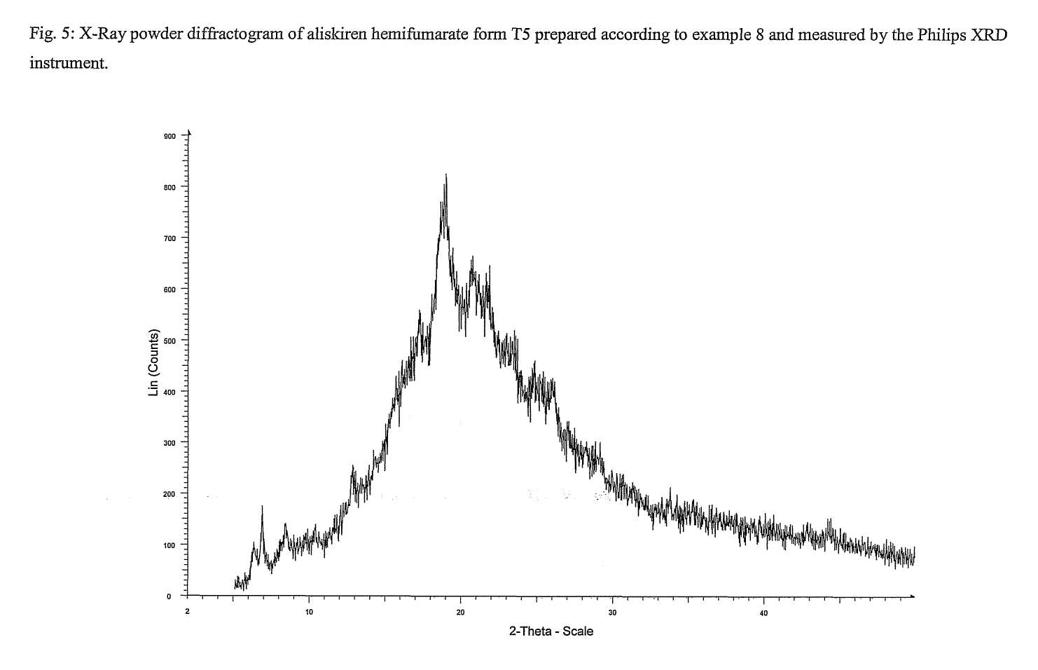

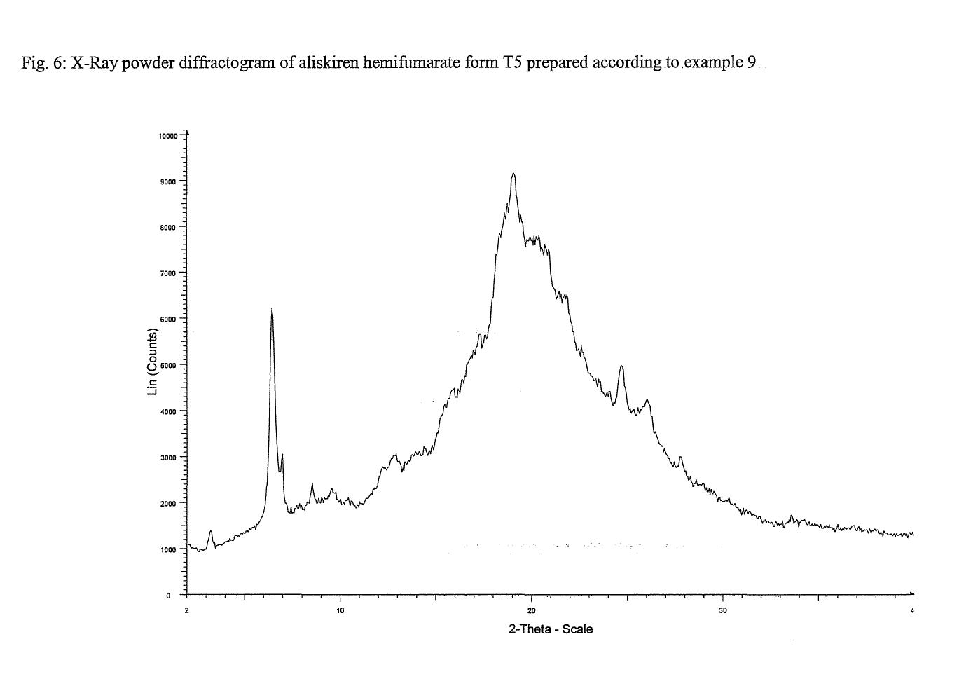

The present invention also provides a crystalline form of aliskiren hemifumarate, designated form T5, characterized by an XRPD pattern with peaks at 6.4, 6.9, 8.5, 19.0 and 24.7±0.3 degrees 2-theta, and optionally additional peaks at 3.2, 12.9 and 17.3±0.3 degrees 2-theta as occur in the X-ray diffractograms depicted in FIGS. 5 and 6.

Aliskiren hemifumarate form T5 can be prepared by a process comprising slurrying amorphous aliskiren hemifumarate in anisole. Preferably, dry anisole is used.

Preferably, form T5 obtained according to the process described above is recovered by solvent decantation followed by evaporation. Evaporation is preferably performed under a flow of nitrogen.

Slurrying is preferably carried out at a temperature of about 20° C. to about 45° C. for a period of about 60 to about 75 hours. More preferably, slurrying is performed at intervals at a temperature in the range from about 20° C. to about 45° C., or about 25° C. to about 35° C., every 2 to 5 hours up to a total of about 70 to about 75 hours. Most preferably, slurrying is done at intervals at about 25° C. to 40° C. every 4 hours up to a total of about 72 hours.

The amorphous aliskiren hemifumarate described in any of the processes described above may be obtained according to any method known in the art, such as the ones described in the co-pending application WO 20090064479 or according to Example 1 of the present application. Preferably, the amorphous aliskiren hemifumarate is prepared according to example 1 (a or b) of the present application, wherein the amorphous aliskiren hemifumarate is recovered by evaporation from a solution of aliskiren hemifumarate in ethanol. The aliskiren hemifumarate solution used to prepare the amorphous form may be obtained from form VIII or from a mixture of aliskiren form I, form VIII and the amorphous form.

Aliskiren hemifumarate form T5 can be also prepared by a process comprising combining aliskiren base, fumaric acid and anisole and removing the solvent from the reaction mixture to obtain aliskiren hemifumarate form T5.

Preferably, aliskiren base is used at a ratio of about 1:3 (w/V) to about 1:12 (w/V) of grams of aliskiren base to milliliters of anisole. More preferably, the ratio is about 1:5 to about 1:10 and most preferably, the ratio is about 1:10 (w/V).

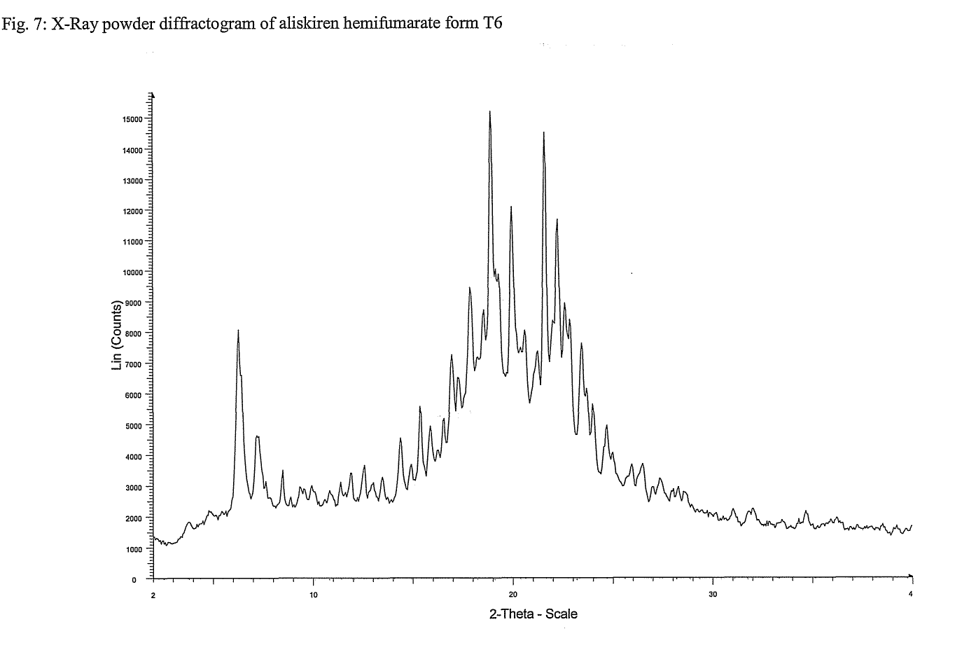

The present invention also provides a crystalline form of aliskiren hemifumarate, designated form T6 characterized by an XRPD pattern with peaks at 6.3, 7.2, 14.3, 15.4 and 17.0±0.2 degrees 2-theta and optionally additional peaks at 17.9, 18.9, 20.0, 21.6 and 22.2±0.2 degrees 2-theta as are present in the X-ray diffractogram depicted in FIG. 7.

Aliskiren hemifumarate form T6 can be prepared by a process comprising combining aliskiren base, fumaric acid and tetrahydrofuran (THF) and removing the solvent from the reaction mixture to obtain aliskiren hemifumarate fowl T6.

Preferably, aliskiren base is used at a ratio of about 1:3 (w/V) to about 1:12 (w/V) of grams of aliskiren base to milliliters of THF. More preferably, the ratio is about 1:5 to about 1:10 and most preferably the ratio is about 1:10 (w/V).

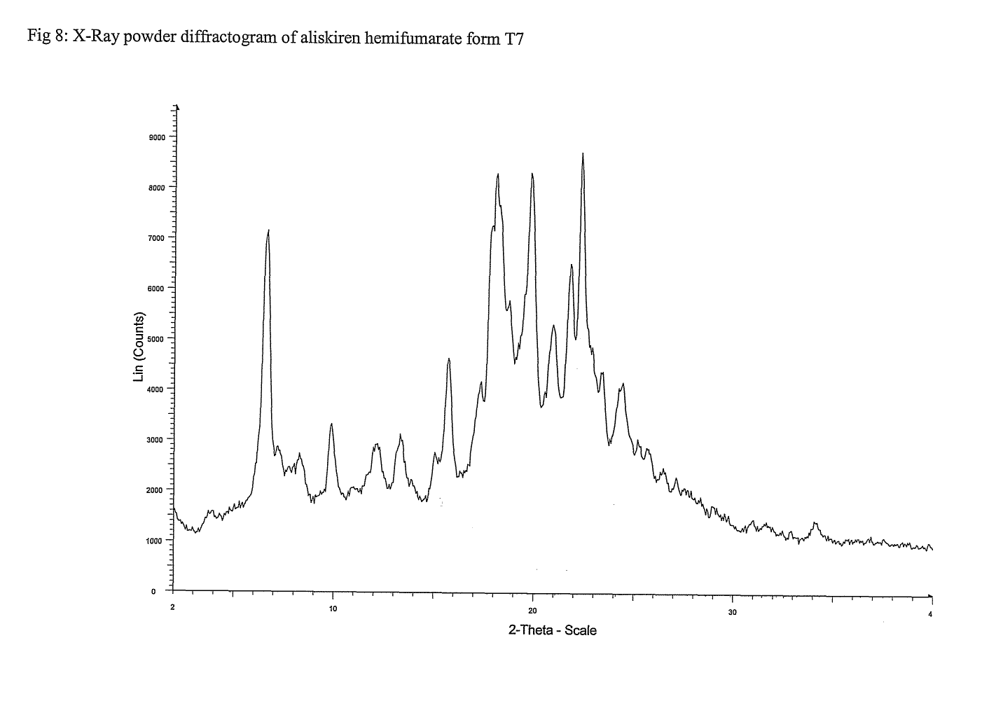

The present invention also provides a crystalline form of aliskiren hemifumarate, designated form T7 characterized by an XRPD pattern with peaks at 6.6, 9.8, 15.7, 19.8 and 22.4±0.2 degrees 2-theta and optionally additional peaks at 12.1, 13.3, 18.1, 21.0 and 21.8±0.2 degrees 2-theta as are present in the X-ray diffractogram depicted in FIG. 8.

Aliskiren hemifumarate form T7 can be prepared by a process comprising combining aliskiren base, fumaric acid and dimethylcarbonate and removing the solvent from the reaction mixture to obtain aliskiren hemifumarate form T7.

Preferably, aliskiren base is used at a ratio of about 1:3 (w/V) to about 1:12 (w/V) of grams of aliskiren base to milliliters of dimethylcarbonate. More preferably, the ratio is about 1:5 to about 1:10 and most preferably, the ratio is about 1:10 (w/V).

Solvent removal in any of the processes described above may be obtained by filtration or evaporation. Preferably, filtration is used.

The present invention also provides a process for preparing amorphous aliskiren hemifumarate comprising dissolving aliskiren hemifumarate in ethanol and evaporating the solvent.

The present invention provides another process for preparing amorphous aliskiren hemifumarate comprising dissolving aliskiren hemifumarate in dichloromethane; adding Diisopropylether; and filtering the obtained precipitate.

The present invention further encompasses (1) a pharmaceutical composition comprising aliskiren hemifumarate crystalline forms T1, T3 or T4, T5, T6, T7 and T8 described above and at least one pharmaceutically acceptable excipient, and (2) the use of aliskiren hemifumarate crystalline forms T1, T3 or T4 described above, for the manufacture of a pharmaceutical composition, wherein the pharmaceutical composition can be useful for the treatment of hypertension.

The pharmaceutical composition of the present invention can be in a solid or a non-solid form. If the pharmaceutical composition is in a non-solid form, the aliskiren hemifumarate in the composition can be present as a solid in the non-solid pharmaceutical composition, e.g., as a suspension, a foam, an ointment, etc.

The pharmaceutical composition can be prepared by a process comprising combining one or more of the above-described aliskiren hemifumarate crystalline forms T1, T3 or T4, T5, T6, T7 and T8 with at least one pharmaceutically acceptable excipient. The aliskiren hemifumarate crystalline forms T1, T3 or T4 T5, T6, T7 and T8 can be obtained by any of the processes of the present invention as described above. The pharmaceutical composition can be used to make appropriate dosage forms such as tablets, powders, capsules, suppositories, sachets, troches and lozenges.

The aliskiren hemifumarate crystalline forms T1, T3 or T4, T5, T6, T7 and T8 of the present invention, particularly in a pharmaceutical composition and dosage form, can be used to treat hypertension in a mammal such as a human, comprising administering a treatment effective amount of the aliskiren hemifumarate in the mammal. The treatment effective amount or proper dosage to be used can be determined by one of ordinary skill in the art, and can depend on the method of administration, the bioavailability, the age, sex, symptoms and health condition of the patient, and the severity of the disease to be treated, etc.

### EXAMPLES

### PXRD Methods

X-Ray powder diffraction data was obtained by methods known in the art using a SCINTAG powder X-Ray diffractometer, Bruker X-Ray diffractometer and Philips X-Pert MPD diffractometer.

Forms T1, T3 and T4 were analysed using SCINTAG powder X-Ray diffractometer model X'TRA equipped with a solid-state detector at a CuKα radiation. A round aluminum sample holder with zero background was used. The scanning parameters included: range: 2-40 degrees two-theta; scan mode: continuous scan; step size: 0.05 deg; and a rate of 5 deg/min. All peak positions are within±0.2 degrees two theta.

Form T5-T8 were analyzed on a Bruker X-Ray powder diffractometer model D8 advance equipped with lynxEye position sensitive detector at a CuKα radiation.

Sample holder: a standard sample holder of PMMA. (In case of low amount of material, standard sample holder of PMMA was used with zero background plate).

Scan range: 2-40°. Step size: 0.05°. Time per step: 5.2 seconds.

Scanning parameters:
Sample: Spin mode, rotation speed: 60 rpm.
Range: 2-40 degrees two-theta.
Scan mode: Continuous scan.
Step size: 0.05°
Time/Step: 0.5 sec.
Divergence slit: 0.982°

Form T5 (FIG. 5) was also analyzed on a Philips X-Pert MPD diffractometer using CuKa radiation. The scanning parameters were: Range: 4-40 deg. 2, continuous Scan, Rate:

FIG. 9 (Form T8)—The peak positions are determined using silicon powder as an internal standard in an admixture with the sample measured. The position of the silicon (111) peak was corrected to be 28.45 degrees two theta.

In the powder XRD measurements taken with silicon mixed with the aliskiren hemifumarate, the peak positions were calibrated using silicon powder as an internal standard in the admixture when the powder XRD of the sample was measured. The position of the silicon (111) peak was corrected to be 28.45 degrees two theta. The positions of aliskiren hemifumarate forms T8 peaks were corrected respectively. No correction was performed on the diffractograms presented in the figures.

Scanning parameters for FIG. 9 (Form T8 with silicon): Range: 2-40 deg. 2 theta, continuous Scan, Rate: 1 degree/minute, Step Size: 0.02. The accuracy of peak positions is defined as +/−0.1 degrees.

### Preparation of Aliskiren Hemifumarate Amorphous (Reference Examples):

### Example 1a

To a 3 L flask was added aliskiren hemifumarate (620 g) and absolute ethanol (4 L). The mixture was stirred at room temperature for complete dissolution and then filtered. The ethanol was evaporated at 30° C. (less than 50 mbar) to obtain a white solid foam. The foam was crushed and dried in a vacuum oven at 35-40° C. (˜10 mbar) for 6 days to give aliskiren hemifumarate amorphous as a white powder (518.3 g, yield 87.8%).

### Example 1b

To a 1 L flask was added aliskiren hemifumarate (20 g, 32.79 mmole form VIII) and absolute ethanol (0.3 L). The mixture was stirred at room temperature for complete dissolution. The ethanol was then evaporated at 30° C. (less than 50 mbar) to obtain a white solid foam. The foam was crushed and dried in a vacuum oven at 30-35° C. (˜10 mbar) for 6 days to give aliskiren hemifumarate amorphous as a white powder.

### Example 2

### Preparation of Aliskiren Hemifumarate Form T1

A suspension of amorphous aliskiren hemifumarate (0.5 g) and 2-methyltetrahydrofuran (2 mL) was stirred for about 72 h at ambient room temperature. The thick paste obtained within a few minutes was analyzed by XRPD and found to be form T1 of aliskiren hemifumarate.

### Example 3

### Preparation of Aliskiren Hemifumarate forms T3 and T4

To a 20 mL vial was added amorphous aliskiren hemifumarate (3 g, 4.9 mmole) and 2-Me THF (20 mL). The almost clear solution was stirred for 5 minutes. A thick colorless gel was obtained. After stirring for an additional 1 hour, a white to colorless paste was obtained to give aliskiren hemifumarate form T3. Drying at 60° C. under reduced pressure overnight gave aliskiren hemifumarate form T4.

### Preparation of Aliskiren Hemifumarate Form T4:

### Example 4

Aliskiren hemifumarate form T1 was dried in a vacuum oven at 55-60° C. (˜10 mbar) overnight to give aliskiren hemifumarate form T4.

### Example 5

To a 20 mL vial was added aliskiren hemifumarate (0.5 g, 0.82 mmole amorphous form), trichloroethane (2 mL) and isopropyl acetate to faun a slurry. The slurry was stirred for 11 days and a paste was obtained to give aliskiren hemifumarate fowl T4. Drying at 60° C. under reduced pressure overnight gave form T4.

### Example 6

To a 20 mL vial was added aliskiren hemifumarate (0.5 g, 0.82 mmole amorphous form), trichloroethane (2 mL) and methyl tert-butyl ether to form a slurry. The slurry was stirred for 11 days, and a paste was obtained to give aliskiren hemifumarate form T4. Drying at 60° C. under reduced pressure overnight gave form T4.

### Example 7

To a 20 mL vial was added aliskiren hemifumarate (0.5 g, 0.82 mmole amorphous form) and methyltetrahydrofuran (2 mL), forming a slurry. The slurry was stirred for 5 minutes to give a gel. Toluene (0.3 mL) was added and stirred for 4 days. A paste was obtained to give aliskiren hemifumarate form T4. Drying at 60° C. under reduced pressure overnight gave form T4.

### Preparation of Aliskiren Hemifumarate Form T5:

### Example 8

Amorphous aliskiren hemifumarate (50 mg) was weighed into a Japanese vial, and anisole (100 μl), which had been dried over 4A molecular sieves, was added to the solid. The vial was then sealed and the resulting slurry was shaken using a Heidolph Titramax 1000 platform, which was linked to a Heidolph Inkubator 1000 as the temperature was cycled between ambient temperature and 40° C. every 4 hours. After a total of 72 hours the sample was removed and the excess solvent was decanted off using a syringe. The residual solid was allowed to dry by evaporation under a flow of nitrogen to give aliskiren hemifumarate form T5. XRD analysis provided the pattern presented in FIG. 5.

### Example 9

To a 20 mL vial was added aliskiren base (0.54 g, 0.97 mmole), anisole (5.4 mL) and fumaric acid (56 mg, 0.48 mmole). The clear solution was stirred overnight. A white paste was obtained; and it was filtered to give aliskiren hemifumarate form T5. XRD analysis provided the pattern as presented in FIG. 6.

### Example 10

### Preparation of Aliskiren Hemifumarate Form T6

To a 20 mL vial was added aliskiren base (0.73 g, 1.32 mmole), THF (7.3 mL) and fumaric acid (76 mg, 0.66 mmole). The clear solution was stirred overnight. A white paste was obtained; it was filtered to give aliskiren hemifumarate faun T6.

### Example 11

### Preparation of Aliskiren Hemifumarate Form T7

To a 20 mL vial was added aliskiren base (0.6 g, 1.09 mmole), dimethylcarbonate (6 mL) and fumaric acid (63 mg, 0.54 mmole). The clear solution was stirred overnight. A white paste was obtained; it was filtered to give aliskiren hemifumarate form T7.

### Example 12

### Preparation of Aliskiren Hemifumarate Form T8

Aliskiren hemifumarate form VIE was delumped by a comill (granulator) and was then placed in a static vacuum oven at 45±5° C. for 48 hours and then the temperature was changed to 60±5° C. for an additional 96 hours. The material was stirred manually occasionally (approximately every 8 hours). The final crystal foam was form T8.

### Preparation of Amorphous Aliskiren:

### Example 13

To a 20 mL vial was added aliskiren hemifumarate (0.5 g, 0.82 mmole form T3), and benzyl alcohol (2 mL). The resulting solution was stirred overnight at room temperature. To the solution was added hexane (1.5 mL) and then MTBE (1.5 mL). It was then stirred for an additional 2 hours. The solvent was taken out by decantation to give amorphous aliskiren hemifumarate.

### Example 14

To a 20 ml vial was added aliskiren hemifumarate (0.5 g, 0.82 mmole faun T3), and methyl formate (2 mL). The resulting solution was stirred overnight at room temperature. To the solution was added hexane (1.5 mL) and then MTBE (1.5 mL). It was then stirred for an additional 2 hours. The solvent was taken out by decantation to give amorphous aliskiren hemifumarate.

### Example 15

To a 20 mL vial was added aliskiren hemifumarate (0.5 g, 0.82 mmole form T3) and DEKALIN© (2 mL). The resulting slurry was stirred overnight at room temperature. The solvent was taken out by decantation to give amorphous aliskiren hemifumarate.

### Example 16

To a 20 mL vial was added aliskiren hemifumarate (0.5 g, 0.82 mmole form T3) and dimethylacetamide (2 mL). The resulting solution was stirred overnight at room temperature. To the solution was added hexane (1.5 mL) and then MTBE (1.5 mL). The resulting mixture was then stirred for an additional 2 hours. The mixture was then filtered to give amorphous aliskiren hemifumarate.

### Example 17

To a 20 mL vial was added aliskiren hemifumarate (0.5 g, 0.82 mmole form T3) and N-methyl-2-pyrrolidone (2 mL). The resulting solution was stirred overnight at room temperature. To the solution was added hexane (1.5 mL) and then MTBE (1.5 mL). The resulting mixture was then stirred for an additional 2 hours. The mixture was then filtered to give amorphous aliskiren hemifumarate.

### Example 18

To a 20 mL vial was added aliskiren hemifumarate (0.3 g, 0.49 mmole. form T3), and amyl alcohol (1.2 mL). The resulting slurry was stirred overnight at 60° C. and then cooled to room temperature to obtain a clear solution. 2 ml of diethyl ether was added and the resulting solution was stirred for an additional 2 hours to precipitate the product. The solvent was taken out of the mixture by decantation to give amorphous aliskiren hemifumarate.

### Example 19

### Preparation of Aliskiren Hemifumarate Form T8

About 0.2 g of form VIII of aliskiren hemifumarate was stored at 70° C. for about 30 minutes. After cooling to room temperature the sample was analyzed by XRPD. The crystal form was found to be fond T8 of aliskiren hemifumarate.

### Example 20

### Preparation of Aliskiren Hemifumarate Form T8

About 0.1g of aliskiren hemifumarate form VIII was stored in a desiccator under relative humidity of 0% at room temperature for 6 days. The sample was then analyzed by XRPD. The crystal form was found to be form T8 of aliskiren hemifumarate.

### Example 21

### Preparation of Amorphous Aliskiren Hemifumarate

To a 1 L glass reactor was added aliskiren hemifumarate (60 g, 98.4 mmole form VIII) and dichloromethane (180 ml). The mixture was stirred at room temperature until complete dissolution and cooled to 10° C. Diisopropylether (750 ml) was added dropwise to this solution at 10° C. (over 2 hours) to obtain a white solid in suspension. At the end of the addition, the suspension was stirred for an additional 30 minutes. The solid was then filtered and dried in a vacuum filter dryer at 40° C.-60° C. (˜10 mbar) for 12 hours to give aliskiren hemifumarate amorphous as a white powder (55 g, yield 95%).

1. A process for preparing amorphous aliskiren hemifumarate comprising dissolving aliskiren hemifumarate in dichloromethane; adding diisopropylether; and filtering the obtained precipitate.

2-5. (canceled)

6. Crystalline Aliskiren hemifumarate Form T8, characterized by a PXRD pattern having peaks at 13.3°, 14.1°, 18.7°, 19.4° and 20.4°±0.2 degrees 2-theta and free from a peak at 20.1°±0.2 degrees 2-theta.

7. The Aliskiren hemifumarate Form T8 according to claim 6, further characterized by a powder PXRD pattern as shown in the following figure:

[symbol]

8. Crystalline Aliskiren hemifumarate Form T1, characterized by an XRPD pattern having peaks at 4.3, 8.5, 12.8, 21.4 and 24.1±0.2 degrees 2-theta.

9. The Aliskiren hemifumarate Form T1 according to claim 8, further characterized by additional XRPD peaks at 5.3, 8.0, 10.3, 15.5 and 19.4±0.2 degrees 2-theta.

10. The Aliskiren hemifumarate Form T1 according to claim 8, further characterized by an X-ray diffractogram as depicted in FIG. 1.

11. Crystalline Aliskiren hemifumarate Form T3, characterized by an XRPD pattern having peaks at 5.1, 9.1, 10.3, 15.5 and 18.3±0.3 degrees 2-theta.

12. The Aliskiren hemifumarate Form T3 according to claim 11, further characterized by additional XRPD peaks at 5.4, 18.8, 20.8, 22.9 and 23.9±0.3 degrees 2-theta.

13. The Aliskiren hemifumarate Form T3 according to claim 11, further characterized by an X-ray diffractogram as depicted in FIG. 2.

14. Crystalline Aliskiren hemifumarate Form T4, characterized by an XRPD pattern having peaks at 5.6, 9.4, 10.6, 15.4 and 16.2±0.2 degrees 2-theta.

15. The Aliskiren hemifumarate Form T4 according to claim 14, further characterized by additional XRPD peaks at 7.5, 8.9, 18.3, 20.4 and 24.6±0.2 degrees 2-theta.

16. The Aliskiren hemifumarate Form T4 according to claim 14, further characterized by an X-ray diffractogram as depicted in FIG. 3 or FIG. 4.

17. A pharmaceutical composition comprising one or more crystalline Aliskiren hemifumarate forms selected from the group consisting of Form T1, Form T3, Form T4, and Form T8, and at least one pharmaceutically acceptable excipient.

18. A method of treating hypertension comprising administering one or more crystalline Aliskiren hemifumarate forms selected from the group consisting of Form T1, Form T3, Form T4, and Form T8 to a patient suffering from hypertension.
